(19) United States
(12) Patent Application Publication
(10) Pub. No.: US 2012/0021497 A1
(43) Pub. Date: Jan. 26, 2012

(54) HYDRODYNAMIC EXTRACTION OF OILS FROM PHOTOSYNTHETIC CULTURES

(76) Inventor: Mario C. Larach, San Diego, CA (US)

(21) Appl. No.: 13/124,388

(22) PCT Filed: Oct. 14, 2009
(86) PCT No.: PCT/US2009/060722

§ 371 Date: Oct. 11, 2011

Related U.S. Application Data
(60) Provisional application No. 61/105,190, filed on Oct. 14, 2008.

(51) Int. Cl. C12N 1/12

(52) U.S. Cl. 435/257.1

(57) ABSTRACT

The presently described invention relates to the method of extraction of oils from photosynthetic cultures using hydrodynamic cavitation technology for the production of biofuels or other products. This method is referred to herein as Hydrodynamic extraction.

### RELATED APPLICATIONS

This application claims the benefit of priority of U.S. Provisional Application No. 61/105,190, which is hereby incorporated by reference in its entirety.

### TECHNICAL FIELD

The disclosed invention relates to the method of extraction of oils from photosynthetic cultures using hydrodynamic cavitation technology for the production of biofuels or other products.

### BACKGROUND ART

Microalgae and other photosynthetic cultures produce and store lipids, fatty acids, monoglycerides, and diglycerides that can make up a significant percentage of their total ash free dry weight. The hydrocarbons produced by microalgae and other photosynthetic cultures often form oils. Microalgae contain a wide variety of oil lipids, which include membrane-bound polar lipids and non-polar lipids that also encompass free fatty acids and fatty acids. Lipid fractions as high as 70-85% have been reported in some microalgae.

Microalgae oil plays an essential nutritional role in the marine animal world. A 60 ton-blue-whale may have 2 tons of microalgae plankton in its gut for nutrition. The oil contents of whale, fish, and shark-liver oil are the condensates of oil droplets originally stored in the microalgae cells. For marine culture of zooplankters, larval shrimp, and juvenile oysters the aquaculture industry has long used microalgae as a food source not only because of their characteristically high lipid and fatty acid content, but also because of their abundance of certain polyunsaturated fatty acids (PUFAs) essential to the marine animal diet.

In addition to cultivating microalgae as an oil-rich nutritional source for aquaculture, oils derived from cultivated microalgae are used for pharmaceutical, nutraceutical, and cosmetic purposes. Products made from microalgae oils command a very large per acre revenue (over $600,000 per acre per year). With such high profit margins production cost efficiencies and new technologies are not aggressively pursued.

Another potential application of oils derived from cultivated microalgae is for use in the production of biofuels, which are fuels suitable for burning in standard internal combustion engines that are derived from biological sources. In fact most of the fossil fuels extracted from the seas (both former and current) are derived from the oils synthesized and stored by the microalgae of the past ages. With the increasing demand and shrinking supply of fossil fuels, there is a present need for alternative fuels and the promise of biofuels derived from microalgae oils have recently been a source of major interest and investments.

In comparison to the pharmaceutical, nutraceutical, or cosmetic products made from microalgae oils, biofuels from microalgae have a much lower per acre revenue (under $30,000 per acre per year) and thereby highly sensitive to operating cost efficiencies. Current technologies used in the core processes of microalgae oil production are inefficient and have high operating costs. New technologies must be developed to deliver cost-efficient production processes before microalgae biofuels become commercially economic.

An important process step in harvesting microalgae oil is extraction. Extraction is the process of removing the oils from the microalgae cells. Microalgae oils are extracted through a wide variety of methods. Current extraction technologies are costly and do not lend themselves to an efficient and cost effective production systems. Estimates of the current costs to extract oil from microalgae vary, but are likely to be around $1.80/kg or $2.91/liter ($11.00 gallon)

Mechanical pressing is the simplest method of extraction. Because different strains of microalgae vary widely in their physical attributes, various press configurations (screw, expeller, piston, etc) work better for specific microalgae types. Using typical methods, microalgae are harvested, dried, and then can be “pressed” out with an oil press. A press can extract between 70-75% of the oils out of microalgae. Often, mechanical pressing is used in conjunction with chemical solvents. While simple in design, this is a highly energy intensive and extraction efficiency is low.

Chemical solvent extraction, used alone or in combination with other methods, is another common methodology for extracting microalgae oils. Oils from the algae are extracted through repeated washing, or percolation, with an organic solvent under reflux in special glassware. Benzene and ether have been used, but a more popular chemical for solvent extraction is hexane, which is widely used and is less expensive. A downside to using solvents for oil extraction is the inherent dangers involved in working with the chemicals. Care must be taken to avoid exposure to vapors and direct contact with the skin, either of which can cause serious damage. For example, benzene is classified as a carcinogen. Additionally, chemical solvents also present the problem of being an explosion hazard.

Hexane solvent extraction can be used in isolation or it can be used along with the mechanical press method. After the oil has been extracted using a mechanical press, the remaining pulp can be mixed with cyclohexane to extract the remaining oil content. The oil dissolves in the cyclohexane, and the pulp is filtered out from the solution. The oil and cyclohexane are separated by means of distillation. These two stages (cold press & hexane solvent) together will be able to derive more than 95% of the total oil present in the microalgae.

Another extraction method is enzymatic extraction which uses enzymes to degrade the cell walls with water acting as the solvent, making fractionation of the oil much easier. The costs of this extraction process are estimated to be much greater than hexane solvent extraction. The enzymatic extraction can be supported by ultrasonication. The combination “sonoenzymatic treatment” causes faster extraction and higher oil yields.

Ultrasonic-assisted extraction, a branch of sonochemistry, can greatly accelerate extraction processes. Using an ultrasonic reactor, ultrasonic waves are used to create cavitation bubbles in a solvent material, when these bubbles collapse near the cell walls, it creates shock waves and liquid jets that cause those cells walls to break and release their contents into the solvent. A variant of ultrasonic extraction is electrostatic shockwave extraction where cavitation bubbles are created by an ultra-high electric impulse rather than by an acoustic impulse. Sonochemistry can be done wet or dry. If done wet the water will need to be extracted from the mash before extraction of oils with a solvent.

Osmotic shock is yet another method used for extraction. Osmotic shock is a sudden reduction in osmotic pressure that can cause cells in a solution to rupture. Osmotic shock extraction can be performed by taking a high saline growth medium, harvesting to a sludge, and then dumping the sludge into distilled water which will burst nearly all the cells and then the oil can be skimmed off the surface.

Another method of extraction is supercritical fluid extraction. In supercritical fluid/CO₂ extraction, CO₂ is liquefied under pressure and heated to the point that it has the properties of both a liquid and a gas, this liquefied fluid then acts as the solvent in extracting the oil. This method requires special equipment for containment and pressure in the supercritical fluid/CO₂ extraction. Supercritical fluid extraction does not need to be absolutely dry as by varying the pressure and temperature one can fractionate the sample being extracted.

Electroporation is yet another extraction method. With electroporation, ultra high electric impulses are directed toward the microalgae perforating the cell walls to release the oil contents.

All these extraction methods are too costly and complex for commercial use on a large scale. In addition these extraction methods are static batch processes and do not lend themselves to a cost efficient system. These extraction methods are also dependant on the effectiveness of the preceding post-cultivation processes of harvesting and de-watering which are required for removing the microalgae from its growth medium and increasing the microalgae cell density by removing most of the water content in order to prepare the microalgae for current oil extraction methods; current technologies used for harvesting and de-watering processes have very high operating costs. In summary, current extraction methods are not suitable for the low-cost production of biofuels from microalgae oils on a commercial scale. To enable low-cost microalgae biofuel production an extraction process needs to have a low capital cost, ultra low operating costs, and lend itself to an integrated, economical, and continuous production system on a commercial scale.

### SUMMARY OF THE INVENTION

The presently described invention relates a method for the continuous extraction of hydrocarbons from photosynthetic organism, and the apparatus for performing the method. In a preferred embodiment the method comprises the step of applying hydrodynamic cavitation to a continuous flow of microalgae in its growth medium to rupture the cell walls and extract the microalgae oil.

Another preferred embodiment of the invention comprises the step of applying hydrodynamic cavitation to a continuous flow of microalgae in a fluid medium to rupture the cell walls and extract the microalgae oil.

As part of the hydrodynamic cavitation process the growth medium or fluid medium used is sterilized for reuse.

The presently invention relates to a method of rupturing a microalgae cell wall, comprising providing a continuous flow of a fluid medium comprising one or more microalgae to a hydrodynamic cavitation device; applying hydrodynamic cavitation in sufficient quantity to rupture one or more microalgae cells, whereby microalgae oil is released from the microalgae into the medium; and extract the microalgae oil from the medium. In one embodiment, the method further comprises the step of dewatering the medium. In one embodiment, the photosynthetic organism is a diatom, such as a Chaetoceros species. In another embodiment of the invention, the hydrodynamic cavitation is applied using a multi-stage hydrodynamic cavitation reactor, and in another, the hydrodynamic cavitation is applied using a magnetic impulse cavitation reactor. In another aspect of the invention, the processed medium after hydrodynamic cavitation is separated into components comprising microalgae oil, microalgae cell walls, and the processed fluid medium. The separated components can be further processed into a biofuel, such as biodiesel, and such further processing can comprise one or more additional rounds of hydrodynamic cavitation to produce transesterification. The processed medium can also be recycled for use as microalgae cultivation medium, and the processed medium is subjected to one or more additional rounds of hydrodynamic cavitation. Another embodiment of the invention relates to a biofuel produced by the disclosed methods, where a particular biofuel that can be prepared is a biodiesel.

### DETAILED DESCRIPTION OF THE INVENTION

The presently described invention relates to continuous production and extraction methods for harvesting hydrocarbons from microalgae and other photosynthetic cultures. Extraction methods that enable a continuous production process are preferred over static batch processes because continuous production methods significantly reduce the cost of producing finished biofuels or other products. Hydrodynamic cavitation is a preferred method of extracting hydrocarbons of interest from microalgae and other photosynthetic cultures.

The choice of extraction technologies will depend largely on the nature of the photosynthetic organism in culture. Organic-wall microalgae are very suitable to hexane solvent and enzymatic extraction. Living silica-wall microalgae (diatoms) however render their own cell walls extremely insoluble. In addition silica creates a physically strong and chemically inert protective covering since the cell walls cannot be attacked enzymatically. Silicon uptake and deposition by diatoms involves less metabolic energy expenditure than formation of equivalent organic walls resulting in faster growth rates than their organic-wall counterparts which makes diatoms attractive for high yield cultivation. Current extraction technologies however greatly inhibit diatom cultivation for oil production and favor organic wall microalgae cultivation. The silica cell structure of diatoms requires the use of cell disruption technologies that liberates the oils from the cultured organisms and allows for the isolation of the high-quality silica (diatomite). A preferred cell disruption technology is hydrodynamic cavitation which can be applied effectively to both organic- and silica-wall photosynthetic organisms. Extraction results for different microalgae species at various volume densities indicate that hydrodynamic cavitation achieves near theoretical maximum extraction volumes (see Table 1).

TABLE 1
Extracted Volume vs. Theoretical Maximums

| | Density Volume | | |
|---|---|---|---|
| Microalgae Species | 1.0% | 3.0% | 5.0% |
| Chaetoceros | 96% | 98% | 97% |
| Chlorella | 93% | 95% | 96% |
| Scenedesmus obliquus | 92% | 93% | 94% |
| Botryococcus braunii | 92% | 94% | 95% |

Hydrodynamic Cavitation

Cavitation is the formation of partial vacuums in a liquid by a swiftly moving solid body such as a propeller or by high-intensity sound waves. The partial vacuums are used to rupture the photosynthetic organisms. A variety of examples of hydrodynamic cavitation devices are known in the art. Examples of suitable devices include U.S. Patent Application No. 2009/0192159, as well as U.S. Pat. Nos. 6,279,611, 6,365,555, 6,846,365, 6,935,770, 7,086,777, 7,207,712, and 7,338,551, all of which are hereby incorporated by reference in its entirety.

In a preferred embodiment, a device for creating hydrodynamic cavitation in a fluid is utilized. Typically, the device includes a flow-through chamber having various portions and a plurality of baffles within one of the downstream portions of the chamber. One or more of the baffles is configured to be movable into an upstream portion of the chamber to generate a hydrodynamic cavitation field downstream from each baffle moved into the upstream portion of the chamber.

In another preferred embodiment, a device for creating hydrodynamic cavitation is utilized in which case the device creates hydrodynamic cavitation of the fluid stream by applying magnetic impulse. Magnetic impulse hydrodynamic cavitation offers a more uniform cavitation distribution over flow through chambers hydrodynamic cavitation for processing of liquids in a turbulent flow.

Cavitation (the formation, growth, and implosive collapse of gas or vapor-filled bubbles in liquids) can have substantial chemical and physical effects. While the chemical effects of acoustic cavitation (i.e., sonochemistry and sonoluminescence) have been extensively investigated during recent years, little is known about the chemical consequences of hydrodynamic cavitation created during turbulent flow of liquids.

Hydrodynamic cavitation is the formation of cavitation bubbles and cavities within a liquid stream or at the boundary of the streamlined body resulting from a localized pressure drop in the liquid flow. If, during the process of movement of the liquid, the pressure at some point decreases to a magnitude under which the liquid reaches a boiling point for this pressure (“cold boiling”), then a great number of vapor-filled cavities and bubbles are formed. These vapor-filled cavities and bubbles are called cavitation cavities and cavitation bubbles. Insofar as the vapor-filled bubbles and cavities move together with the flow, they then move into the elevated pressure zone. Then, almost instantaneously, vapor condensation takes place in the cavities and bubbles, and they collapse, creating very large pressure impulses. The magnitude of the pressure impulses within the collapsing cavitation bubbles may reach 150,000 psi. The result of these high-pressure implosions is the formation of shock waves that emanate from the point of each collapsed cavitation bubble. Such high-impact loads result in the breakup of any medium found near the collapsing cavitation bubbles. Collapse of a cavitation bubble near the boundary of phase separation of a liquid-solid particle in suspension results in the breakup of the suspension particles: A dispersion process takes place. Collapse of a cavitation bubble near the boundary of phase separation of a liquid-liquid type results in the breakup of drops of the disperse phase: Cavitation process takes place. Thus, the use of kinetic energy from collapsing cavitation bubbles and cavities is used in the described cavitation process to extract the oils from microalgae and to sterilize the growth medium for reuse.

The following is a description of one embodiment of a suitable cavitation device. As described in the art, a suitable cavitation device or apparatus is capable of producing appropriate bubbles that produce the cavitation effect. All components inside the apparatus are influenced by pressure impulses and advanced hydrodynamic cavitation. Suitable devices stimulate cavitation in hydrodynamic liquids to the point where the end result of processed fluid meets intended emulsification or dispersion criteria.

A particularly preferred embodiment comprises a nano-cavitation generator that utilizes flow-through nano-cavitation technology for producing biodiesel fuel. The nano-cavitation generator will typically include a casing or housing that encloses a flow-through region. The flow-through region will typically comprise an inlet, a flowmeter passage, an intermediate coupling, a reaction chamber having and inlet and an outlet, a reaction chamber cover, and an outlet fitting.

The inlet is a fitting that passes through a portion of the housing. The inlet includes a coupling, whereby an external fluid line is connected to supply a fluid medium or other reaction components to the generator. The inlet is secured to the housing by a retaining ring which holds the inlet in place and provides sealing against leaks. The inlet fitting is connected to a flowmeter passage which includes a flowmeter to measure the flowrate of process fluids. The flowmeter passage is connected to an inlet of the reaction chamber by an intermediate coupling. The connection between the intermediate coupling and the inlet is sealed by an o-ring or other similar structure. The reaction chamber includes a reaction chamber passageway that connects the inlet to the outlet. The reaction chamber cover is connected to the reaction chamber and partially defines the reaction chamber passageway. The outlet fitting of the generator is integral with the reaction chamber cover.

The reaction chamber passageway defines a series of compartments having varying diameters and surface features. In a first preferred embodiment, the series of compartments in sequence from the inlet to the outlet are as follows: inlet compartment, constriction compartment, first reaction compartment, second reaction compartment, final reaction compartment and outlet compartment. A plasmator is positioned in the passageway through the constriction compartment and the first reaction compartment. The configuration and operation of the plasmator will be described below.

A number of the fittings and couplings in the generator are sealed using retaining rings, o-rings or similar structures. The outlet fitting includes an o-ring which forms a water-tight seal in the junction between the outlet fitting or reaction chamber cover and the reaction chamber. Another o-ring forms a water-tight seal in the connection between the reaction chamber and the intermediate coupling. The connection between the intermediate coupling and the flowmeter passage should also be sealed by an o-ring or similar structure, as well as the connection between the inlet fitting and the flowmeter passage. The inlet fitting is retained and sealed against the housing by a retaining ring as described above.

A pressure gauge is positioned in the housing adjacent the reaction chamber. A sensor from the pressure gauge enters the reaction chamber through an access passage. The pressure gauge and sensor are designed to measure the overall pressure in the reaction chamber. As discussed elsewhere, the overall pressure of the reaction chamber should remain at about atmospheric pressure for the generator to operate as intended.

The nano-cavitation generator is static, i.e., contains no moving parts, and is configured for operation at a set fluid velocity and pressure of fluid medium. As described below, the changing of cavity diameters and surface features within the generator causes the generation of cavitational fluid features, i.e., bubbles and localized elevations of temperature and pressure. These localized elevations of temperature and pressure come in the form of eddies of internal temperature and pressure increases. The subsequent collapse of the cavitational bubbles and eddies is such that the outlet liquid stream is homogenized into a stable, ultra-thin emulsion or dispersion.

The inventive device creates nano-cavitation in fluids in a flow-through region between the fluid inlet fitting and the fluid outlet fitting. The flow-through nano-cavitation reactor is a multi-stage process whereby reaction components are manipulated through localized high temperature and pressure impulses and advanced nano-cavitation principles.

Fluid medium enters the generator at the inlet fitting as indicated by flow arrow. As described briefly above, the reaction chamber passageway comprises various compartments of varying diameter and internal surface features such that the cross-sectional area of each changes in relation to the previous compartment, the plasmator can be positioned in the junction between the constriction compartment and the first reaction compartment.

The plasmator can also comprises a constrictor plate having a stem topped by a conical cap. A series of orifices are positioned in the constrictor plate around the stem. The plasmator can be oriented such that the conical cap is centered in the constriction compartment to force the fluid medium to an outer circumferential flow path, i.e., the gap between the wall of the constriction compartment and the edge of the conical cap. The circumferential flow path provides a greatly reduced flow area compared to the open flow area of the inlet compartment. This greatly reduced flow area is thought to lead to the nano-cavitational process described above. The orifices in the constrictor plate 46 provide another point at which the available flow area is greatly reduced and the nano-cavitational process is increased. Finally, sequential compartments in the reaction chamber passageway vary the available flow area and then match the flow area of the inlet fitting.

Processed fluid medium exits the generator at the outlet fitting as indicated by flow arrow. The nano-cavitational process takes place in the reaction chamber, specifically the reaction chamber passageway. The design of the nano-cavitation generator and the theory behind the fluid process taking place is based solely on the static mechanical and physical construction of the device, i.e., the changing diameters, flow areas and cross-sectional areas.

All reactions that take place in the nano-cavitation generator occur at ambient temperature. No agitation or mixing time is required. The nano-cavitational process is run at pressures between 100 psi and 1000 psi, ideally at around 500 psi. The nano-cavitation generator produces an instant reaction process, due to the bonding at the molecular level of free fatty acids (FFA) in the oil or fat with the reaction catalysts. The transesterification process is completed in seconds and finished product is produced immediately. Complete separation of finished biodiesel and glycerin can be achieved within 8-15 minutes via gravitational processes and instantly via centrifugal processes.

While processing vegetable oils, yellow grease, tallow and other animal fats (below 5% percent FFA content) with necessary components in a flow-through nano-cavitator reactor the molecules of FFA are broken apart in micro-explosions. Such micro-explosions result in instant glycerol separation, increased yield, decreased viscosity, increased cetane number, as well as, improvement of power parameters of produced fuel. The inventive generator also increases the effectiveness of any catalysts used in the reaction, as well as, the rate and efficiency of the esterification reaction. Thus, the inventive apparatus not only increases the quality and quantity of pure biodiesel fuel output but also its production rate.

Flow-through nano-cavitation is produced by pressure variations, which are obtained using the geometry of the passageways in the reactor creating variations in velocity and pressure. For example, based upon the geometry of the first preferred embodiment, an interchange of pressure and kinetic energy can be achieved resulting in the generation of cavities as in the case of the orifices in the constrictor plate. The cavitating conditions are generated just after the orifices in the reaction chamber passageway and hence the intensity of the cavitating conditions strongly depends on the number and geometry of the orifices.

When the reaction liquid passes through the orifices, the flow velocities increase due to the sudden reduction in the area offered for the flow, resulting in a decrease in the pressure. In the inventive device, the velocities are increased such that the localized pressure drops below the vapor pressure of the liquid medium under operating conditions and cavities are formed. Such cavities are formed at multiple locations in the reaction chamber. The location of formation strongly depends upon the number of compartments and the configuration of the same in the reaction chamber passageway. However, downstream of the orifices, due to an increase in the flow area, the velocities decrease giving rise to increasing pressures and greater pressure fluctuations. The change in pressure and resultant pressure fluctuations control the different stages of cavitation, namely formation, growth and collapse.

The various devices known in the art makes it possible to accelerate the cavitational reaction causing bubbles to collapse and unite on a molecular level and allow for the production of biodiesel fuel without the addition of large amounts of energy and avoids high-pressure operation. The devices can produce biodiesel fuel using oils or fats. Soaps formed during base catalyzed transesterification are not present after the cavitational transesterification process has been completed, when provided with appropriate conditions. This simplifies the separation of the product phases and prevents the formation of emulsions if a water wash procedure is used for the finished fuel. The amount of water and FFA in the biolipids (oil or fat) are important parameters for the process and should be set using methodologies known to those of ordinary skill in the art to avoid unwanted side products.

Principle of Operation of Cavitation Mixer-Homogenizer Reactor

In its simplest form, basic cavitation consists of the flow-through chamber, with cavitation generator located at the entry. The shape of the cavitation generator significantly affects the character of the cavitation flow and, correspondingly, the quality of dispersing. The optimal cavitation generator design is chosen in a multi-stage cavitator. In general, the cavitation generator works in the following manner. The stream of components to be processed under pressure P1 is charged with the aid of an auxiliary pump at the entry of the flow through chamber. Further, the stream flows around cavitation generator, after which, as a result of the localized pressure constriction, a cavitation cavity is formed. This cavity with its tail part comprises numerous bubbles. The cavitation bubbles flow with the stream to the exit of the flow through chamber into the elevated pressure zone P2. In this zone, the cavitation bubbles collapse, resulting in the dynamic influence on the emulsion drops, particles, or aggregate particles in suspension.

However, in the currently described process a precisely calculated engineered design is used in order to maximize the physical principle of a multi-stage hydrodynamic cavitation operation.

Advantage of Multi-Stage Cavitation

Independent of the physical principle of its operation, the particle size achieved is dependent on one primary parameter in the process of dispersion—the level of energy dissipation in the cavitation reactor and cavitation pump. The higher the level of energy dissipation in the cavitator chamber of the reactor, the smaller the particle size that can be achieved with any given medium.

The preferred multi-stage hydrodynamic cavitation reactor can achieve the smallest particle sizes. The level of energy dissipation in a cavitation reactor is mainly dependent on three vital parameters in the cavitation bubble field: the sizes of the cavitation bubbles, their concentration volume in the disperse medium, and the pressure in the collapsing zone. Given these parameters, it is possible to control the cavitation regime in the reactor and achieve the required quality of dispersion. These parameters are proprietary information.

In the above examples, the volume concentration of cavitation bubbles was on the order of 10%, which is at the low end of the concentration levels normally achieved in a cavitation reactor. By changing the type of cavitation in the reactor, it is possible to change the volume concentration of bubbles in the field from 10 to 60%, and their sizes from 10 to 1000 μm. The very high levels of energy dissipation produced during the collapse of a large number of cavitation bubbles allows the cavitation mixing pump and multi-stage hydrodynamic reactor to produce a very small particle size and very uniform particle size distribution. The results are produced at 500 psi operating pressures, which makes the equipment safe for a daily processing operation.

Magnetic impulse hydrodynamic cavitation creates cavitation bubbles in a turbulent flow of liquid by applying magnetic impulses which create the cavitation bubbles. Pressures created through magnetic impulse hydrodynamic cavitation are similar to those obtained through those created in flow-through baffle hydrodynamic cavitation devices, but the distribution of the cavitation is more uniform and predictable.

For the purposes of this patent, hydrodynamic cavitation is used to refer to hydrodynamic cavitation created in a continuous flow of liquid—whether created by a flow-through baffle device, a magnetic impulse device, or other similar device capable of creating hydrodynamic cavitation in a turbulent continuous flow of liquid without any moving parts.

Hydrodynamic Extraction

Preferably, the hydrodynamic cavitation technology described here is used to extract the oils produced by the cultivated photosynthetic organism. An advantage of this technology is that it eliminates the need for dewatering steps required in other extraction processes. In one embodiment, after harvest, the harvested medium is directly subjected to hydrodynamic cavitation which disrupts the microalgae cell structure and extracts the oils from the microalgae cells. The resulting medium consisting of microalgae oil, microalgae cell biomass, and the harvested medium is flowed through to a separation process for separation. After separation the harvested medium can then be reused.

Another advantage of this technology is that it eliminates the need for harvesting steps. In another embodiment, a significant portion of the growth medium is directly subjected to hydrodynamic cavitation which disrupts the microalgae cell structure and extracts the oils from the microalgae cells. The resulting medium consisting of microalgae oil, microalgae cell biomass, and the harvested medium is flowed through to a separation process for separation. After separation the harvested medium can then be reused.

The oils and biomass produced from a first round of hydrodynamic cavitation can be subjected to subsequent rounds of hydrodynamic cavitation.

Hydrodynamic extraction enables the production of low-cost biofuels from microalgae oils because it is easily integrated into an economic and continuous process. The cost of hydrodynamic extraction using a 10 gallon/minute reactor is approximately $0.002 per gallon of fluid processed which is several orders of magnitude smaller than the alternative combined costs of harvesting, de-watering, and existing extraction technologies. New higher flow-rate reactor designs will significantly bring down the costs. Furthermore hydrodynamic extraction does not require the addition and subsequent removal of costly additives or chemicals. Hydrodynamic extraction also enhances the adoption of diatoms for microalgae oil production.

Biofuel Products

Following extraction and processing, the oil, fats, fatty acids, triglycerides, etc. harvested from the microalgae and other photosynthetic organisms can be proceed to a variety of different useful products. For example, biodiesel can be produced from the products extracted from the cultivated organisms using standard techniques well know to those of ordinary skill in the art. For example, the production of biodiesel (fatty acid methyl esters) is well understood in the art. A discussion of such methods is provided in U.S. Patent Application No. 20090071064, which is hereby incorporated by reference.

According to some embodiments of the present invention, microalgae lipids are harvested and converted to biodiesel using transesterification. The hydrodynamic cavitation devices described herein are effective to perform this conversion. In a particular embodiment, harvested lipids, etc., are subjected to further rounds of hydrodynamic cavitation to achieve the desired result. Further, after the biodiesel has been produced, it can be readily, energy-efficiently, and economically separated from the other chemicals in the reactor effluent using equipment common in the chemical industry.

Microalgae and Other Photosynthetic Organisms

The phrase “microalgae and other photosynthetic organism,” as used herein, includes all algae capable of photosynthetic growth as well as photosynthetic bacteria. Eukaryotic algal strains are preferred for use with the disclosed methodology. Example include Botryococcene sp., Chlorella sp., Gracilaria sp., Sargassum sp., Spirolina sp., Dunaliella sp. (e.g., Dunaliella tertiolecta), Porphyridum sp., and Plurochrysis sp. (e.g., Plurochrysis carterae). Diatoms, such as Chaetoceros sp. are particularly preferred algal strains for use with the presently described invention. These terms may also include organisms modified artificially or by gene manipulation.

Chaetoceros is particularly well suited for use with the presently described invention. There are over 400 species and subspecies known throughout the world. The growth rate of this organism is rapid, with 4 doubling per day, which permits cultures to be grown quickly. These organisms are known to have broad tolerances to temperature and salinity. Chaetoceros is also known to have a favorable lipid content and thus do not require manipulation to produce high quantities of oil.

Cultivation

The organisms selected for culture can be grown in open or closed systems. Open systems are preferred because they require less energy for maintenance and are typically more stable than closed systems. A preferred culture method for maintaining a dominant strain in culture using an open system is described in U.S. Pat. No. 6,673,592, which is hereby incorporated by reference.

Briefly summarized, the cultivation system comprises a container for holding a culture medium. The culture medium includes an initial aqueous solution and a seed stock of photosynthetic organism. The initial aqueous solution is prepared such that optimal conditions for culturing photosynthetic organism of interest are established. Once the optimal conditions are established, the aqueous solution is inoculated with a seed stock of photosynthetic organism. The resulting culture medium is pH controlled in a set range. A light source, preferably the sun, delivers light and heat to the culture medium, facilitating the growth of the photosynthetic organism culture. Periodically, a percentage of the photosynthetic organism culture medium is harvested. The harvested medium is replaced with a non-sterile medium, such as seawater. Alternatively the harvested medium can be replaced by culture medium from which the photosynthetic organisms have been harvested using the hydrodynamic methods disclosed herein. The method is continually repeated, thereby providing for uninterrupted harvests.

Optimal conditions for culturing a selected photosynthetic organism are typically established in the aqueous medium. Optimal conditions are those that allow a seed stock of photosynthetic organism to grow and outcompete predators, contaminants and other potential scavengers. Creating such a medium allows for the mass production of photosynthetic organism outdoors and under non-sterile conditions. Preferably, optimal conditions are attained in the aqueous medium by initially adjusting the concentrations of some or all of the following constituents: nitrogen, phosphorous, vitamin B₁₂, iron chloride, copper sulfate, silicate and Na₂EDTA. The pH of the culture medium is monitored, with adjustments, such as carbon dioxide treatments, performed to maintain the pH at a desired level.

In a preferred embodiment, the present system is used for culturing Chaetoceros sp. as the photosynthetic organism. The container holds an aqueous medium having the following starting characteristics: a carbon dioxide controlled pH of about 8.2, a starting nitrogen concentration of at least 3.0 mg N/liter, a starting phosphorous concentration of at least 2.75 mg P/liter, a starting vitamin B₁₂ concentration of at least 5 micrograms/liter, a starting iron chloride concentration of at least 0.3 mg/liter, a starting copper sulfate concentration of at least 0.01 mg/liter, a starting silicate concentration of at least 10 mg SiO₂/liter, and a Na₂EDTA concentration of 5 mg/liter. The medium is inoculated with a seed stock of Chaetoceros sp. photosynthetic organism and exposed to direct sunlight. The photosynthetic organism grows in the open environment and is periodically and continuously harvested. The harvested volume is replaced with a new seed stock of Chaetoceros sp. photosynthetic organism and culturing is repeated.

While any light source may be used in the present system, culturing the photosynthetic organism under full strength sunlight is the most economical option.

A percentage of the culture is periodically harvested. Preferably, about 60, 70, 80, 90, 95, or 99% of the culture volume is harvested at the conclusion of each period. In preferred embodiments of the present system and method, the culture is harvested once a day, or approximately once every twenty-four hours. As sterile conditions are not required, the harvested volume is readily replaced with non-sterile seed stock of photosynthetic organism, such as seawater. Alternatively, the harvested volume can be replaced by medium subjected to hydrodynamic cavitation. Replaced of the harvested volume with treated medium is advantageous, particularly when some portion of the organisms present in the treated medium remain viable. The volume is preferably manually harvested or harvested using any acceptable harvesting machine or apparatus.

The container, which may have any acceptable dimensions and be constructed of any acceptable material, and preferably has an open top. Preferably large tanks are used as the containers. The tanks may be positioned above ground to permit sunlight to be passed through the sides of the containers. Alternatively, the tanks may be positioned within the ground. A transparent, light-passing cover may be positioned over the open top. In one embodiment, the cover is removably positioned over the open top.

By culturing photosynthetic organism in the optimal conditions, the production of large quantities of photosynthetic organism is possible in a cost effective manner. A single container is situated in an outdoor environment such that the contents of the container are directly exposed to natural light. No artificial light sources or additional transfer tanks are needed. Contaminants and predators are not a problem, as the established media conditions allow the photosynthetic organism to outcompete and overcome unwanted or detrimental species.

By establishing the optimal culture conditions for the photosynthetic organism, the present system provides for an environment where the photosynthetic organism out competes other species of photosynthetic organism from the culture. That enables the photosynthetic organism to be cultured continuously in large, outdoor containers using natural light. The need for labor intensive and costly systems designed to exclude other species from the culture is eliminated. The use of natural light greatly decreases the costs and problems associated with artificial lights.

The following examples are offered to illustrate but not to limit the invention.

### Example 1

### Hydrodynamic Extraction of Oil from Harvested Chaetoceros Microalgae

Chaetoceros sp. is a microalgae diatom species that is particularly suited for fuel production because of desirable growth rates, growing conditions, and oil profile (e.g., lipid composition, lipid concentration as a percentage of mass).

Each day 90% of the culture volume was removed and stored in a harvesting tank. The culture in the harvesting tank was circulated through a foam fractionator column from evening until morning. Air was bubbled upward through the column from the bottom creating foam at the surface of the water that contained concentrated photosynthetic organisms. This foam was collected from the surface of the water. This foam upon condensing into a liquid contained approximately 3% dry matter content.

This harvested medium consisting of 10% of the culture volume with a 3% dry matter content was then directly flowed into a hydrodynamic cavitation reactor that processed 10 gallons per minute at 500 psi operating pressure for Hydrodynamic Extraction. Three hundred and Twenty (320) liters were processed in under 9 minutes. Total processing cost was $0.17.

The hydrodynamic extraction extracted over 98% of the Chaetoceros sp. estimated ash free dry weight oil content and produced over 2.9 liters of microalgae oil at a cost of $0.06 per liter of oil ($0.22 per gallon of oil). This compares to $2.91/liter for extraction using current technologies, not including the cost of the required de-watering steps.

The medium that then flowed out of the hydrodynamic extraction was directly flowed to a separation unit where the microalgae oil, the silica cell walls (diatomite), and the remaining fluid medium were separated. The separated microalgae oil can then be processed for use as biofuel or other product. The separated diatomite can be sold commercially. The separated fluid medium is then directly re-circulated back for cultivation.

1. A method of rupturing a microalgae cell wall, comprising:
providing a continuous flow of a fluid medium comprising one or more microalgae to a hydrodynamic cavitation device;
applying hydrodynamic cavitation in sufficient quantity to rupture one or more microalgae cells, whereby microalgae oil is released from the microalgae into the medium; and
extract the microalgae oil from the medium.

2-14. (canceled)
